(19) United States
(12) Patent Application Publication
(10) Pub. No.: US 2011/0125205 A1
(43) Pub. Date: May 26, 2011

(54) Thermostimulation system including multilayer pads with integrated temperature regulations

(76) Inventor: Mohn Louise, London (GB)

(21) Appl. No.: 12/592,498

(22) Filed: Nov. 25, 2009

(51) Int. Cl. A61F 7/00; A61N 1/36

(52) U.S. Cl. 607/3

(57) ABSTRACT

A thermostimulation system and method. The inventive thermostimulation system is adapted for use with a console for providing electrical currents for thermal and electrical stimulation in response to a first input from an operator via at least one electrical connector. Generally, the inventive thermostimulation system includes at least one inline control system coupled to the console via electrical connector for regulating the currents to an associated thermostimulation pad via a second connector. The pad has a temperature sensor adapted to provide a feedback signal to the inline control system. In more specific embodiments, plural pads and inline control systems are connected to the console. Each inline control system has a first microprocessor for providing heat and stimulation current control for the pad and a second microprocessor for providing overcurrent safety control for the pad. Each inline control system has a display and a patient over-temperature control switch. Each pad has a connector integrated multilayer construction with a heating element implemented with a wire matrix and slots for flexibility. In addition to a temperature sensor, each pad also includes two electrical stimulation contacts having a wire conductor along the length thereof. Each pad is connected to an associated inline control system via a flat connector. Specially designed strain relief grommets are provided on both ends of the flat cable.

### BACKGROUND OF THE INVENTION

1. Field of the Invention

The present invention relates to therapeutic systems. More specifically, the present invention relates to methods and apparatus for providing electrical and thermal stimulation.

2. Description of the Related Art

For a variety of therapeutic applications, several treatment modalities are currently known in the art including electrical stimulation, heat therapy and thermostimulation. Electrical stimulation involves the application of an electrical current to a single muscle or a group of muscles. The resulting contraction can produce a variety of effects from strengthening injured muscles and reducing oedema to relieving pain and promoting healing. Many electrical stimulation systems are limited to two to four channels and therefore allow only two to four pads to be applied to a patient. The pads are usually quite small and typically powered with a battery. This results in the application of a small amount of power and a low treatment depth of the resulting electric field. The shallow depth of the electric field generated by conventional electrical stimulation systems limits performance and patient benefit. Some systems have attempted to address this limitation by applying more current, often from a line or main supply source. However, the small size of conventional electrical stimulation pads is such that on the application of larger amounts of power, i.e. the use of higher currents, patients often report the experience of pain or discomfort.

Heat therapy or thermal stimulation itself is very useful as it has a number of effects such as relaxation of muscle spasm and increased blood flow that promotes healing. However, combination therapy, i.e. the synergistic use of other modalities such as massage, ultrasound and/or electrical stimulation has been found to be more effective than heat therapy alone.

Thermostimulation is one such combination therapy that involves the use of heat therapy and electrical stimulation simultaneously. With thermostimulation, the healing benefits of heat are provided along with the strengthening, toning, pain relieving and healing benefits of electrical stimulation. Moreover, the application of heat has been found effective in that it allows the patient to tolerate higher currents. This yields higher electric fields strengths, greater depths of penetration and therefore, more positive results than could be achieved with electrical stimulation without heat.

Unfortunately, there are several problems associated with conventional thermostimulation systems. One problem is due to poor or inadequate pad design. That is, conventional pads are small, hard and die cut with sharp flat edges. The rectangular shape of the pads does not conform to the natural shape of muscle tissue. In addition, conventional pads tend to exhibit a current fall off over the length of the pad. This limits the performance of conventional pads. Further, the connectors are subject to detachment and therefor often fail to comply with government requirements in certain countries. (See for example EN standard 60601-2-35 for medical electrical devices.)

Further, conventional thermostimulation pads are not waterproof. As a consequence, sweat from the patient combined with the pad gel can cause the stimulation connector and press studs to short directly to the patient, which can result in the patient being shocked or burned.

Moreover, conventional thermostimulation pads are generally inflexible and yield to breakage of the heating element if bent or folded too frequently. More significantly, conventional thermostimulation pads are not designed to detect, measure and/or monitor temperature of the pad when on the patient. Consequently, effective temperature regulation is not provided with conventional thermostimulation systems.

Hence, a need remains in the art for an improved system or method for thermostimulation therapy that is more safe and effective.

### SUMMARY OF THE INVENTION

The need in the art is addressed by the thermostimulation system and method of the present invention. The inventive thermostimulation system is adapted for use with a console for providing electrical currents for thermal and electrical stimulation in response to a first input from an operator via at least one electrical connector. Generally, the inventive thermostimulation system includes at least one inline control system coupled to the console for regulating the currents to an associated thermostimulation pad. The pad has a temperature sensor adapted to provide a feedback signal to the inline control system.

In more specific embodiments, plural pads and inline control systems are connected to the console. Each inline control system has a first microprocessor for providing heat and stimulation current control for the pad and a second microprocessor for providing over-temperature control for the pad. Each inline control system has a display and a button to allow confirmation of temperatures of more than 38 degrees Celsius. Each pad has a connector integrated multilayer construction with a heating element implemented with a wire matrix and slots for flexibility. In addition to a temperature sensor, each pad also includes two electrical stimulation contacts having a wire conductor along the length thereof. Each pad is connected to an associated inline control system via a flat cable. Specially designed strain relief grommets are provided on both ends of the flat cable where they terminate with the pad or inline control system.

The inventive thermostimulation method includes the steps of applying a thermostimulation pad with connector integrated multilayer construction to a patient having a temperature sensor adapted to feedback a temperature signal; coupling the pad to a console via an inline control system; setting the console to generate predetermined electrical currents to the inline control system for thermal and electrical stimulation via a first connector; and regulating the temperature of the pad via the inline control system in response to the predetermined electrical current for thermal stimulation and the feedback temperature signal.

### BRIEF DESCRIPTION OF THE DRAWINGS

FIG. 1 is a perspective view of a typical thermostimulation system implemented in accordance with conventional teachings.

FIG. 2 is a simplified block diagram of a typical thermostimulation electrical system provided within the console of FIG. 1.

FIGS. 3a-3c illustrate the typical conventional thermostimulation pad of FIGS. 1 and 2 in more detail. FIG. 3a is a top view of the pad, FIG. 3b is a sectional side view taken along the line 3b-3b of FIG. 3a, and FIG. 3c is a bottom view of the pad.

FIG. 4 is a simplified perspective view of a thermostimulation system implemented in accordance with an illustrative embodiment of the present teachings.

FIG. 5 shows a perspective bottom view of the pad of FIG. 4.

FIG. 6 is an exploded upside down view of a portion of the pad of FIG. 4 in disassembled relation.

FIG. 6a is a top plan view of the heating element of the illustrative embodiment of the pad of FIG. 4.

FIG. 6b is a magnified view of a portion of the heating element of FIG. 6a.

FIGS. 6c-g show the grommet used in the pad of FIG. 4.

FIG. 6c shows an upper section of the illustrative implementation of the grommet.

FIG. 6d shows a lower section of the illustrative implementation of the grommet.

FIG. 6e is a top view of the grommet.

FIG. 6f is a side view of the grommet.

FIG. 6g is a perspective view of the upper section of the grommet.

FIG. 7 is a perspective side view of the inline control system of FIG. 4 fully assembled.

FIG. 8 is a perspective side view of the inline control system of FIG. 7 disassembled.

FIG. 9 is a sectional side view of the inline control system of FIG. 4 fully assembled.

FIG. 10 below is an electrical block diagram of the inventive system including the control system elements.

FIGS. 11a-c are flow diagrams of the firmware in accordance with an illustrative embodiment of the present teachings.

FIG. 11a is a flow diagram of the firmware executed by the main microcontroller of FIG. 10.

FIG. 11b is a flow diagram of the firmware executed by the safety microcontroller of FIG. 10.

FIG. 11c is a flow diagram of the firmware executed by the main and safety microcontrollers of FIG. 10 for a self-test mode of operation.

### DESCRIPTION OF THE INVENTION

Illustrative embodiments and exemplary applications will now be described with reference to the accompanying drawings to disclose the advantageous teachings of the present invention.

While the present invention is described herein with reference to illustrative embodiments for particular applications, it should be understood that the invention is not limited thereto. Those having ordinary skill in the art and access to the teachings provided herein will recognize additional modifications, applications, and embodiments within the scope thereof and additional fields in which the present invention would be of significant utility.

### Conventional Thermostimulation System

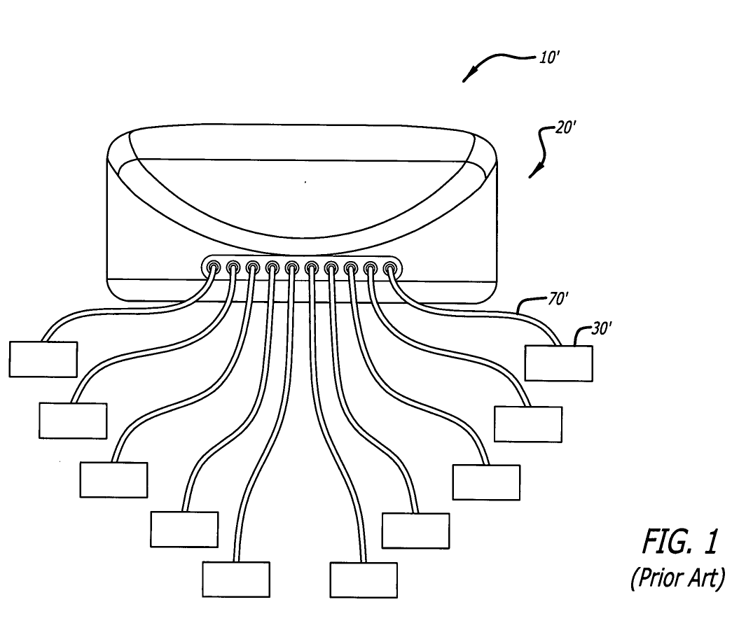

FIG. 1 is a perspective view of a typical thermostimulation system implemented in accordance with conventional teachings. The system 10′ includes a conventional thermostimulation console 20′ and a plurality of thermostimulation pads 30′. The console may be purchased from Ross Estetica of Barcelona Spain. (See http://corporativa.ross.es/rosseng/ross/indexross.htm.)

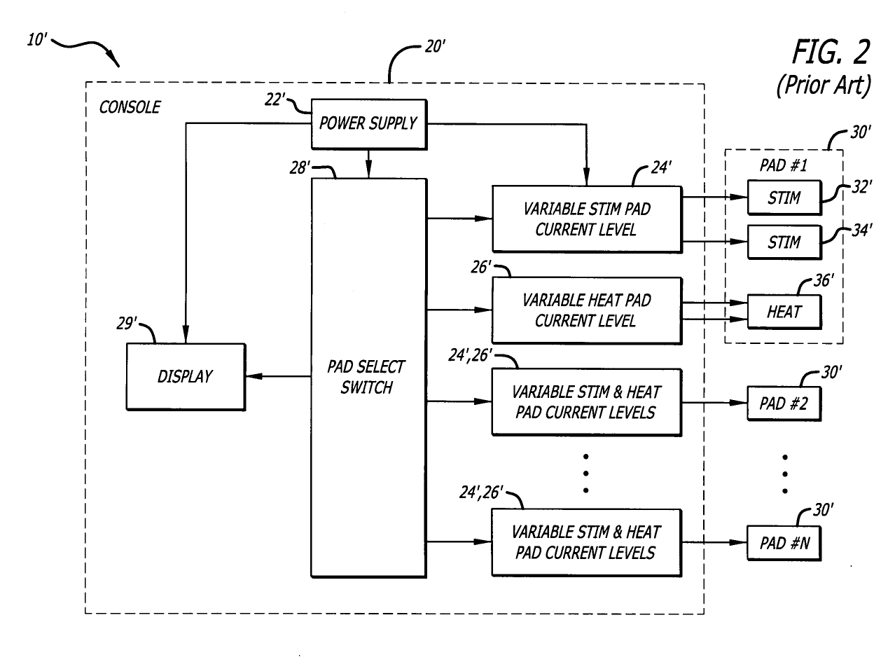

FIG. 2 is a simplified block diagram of a typical thermostimulation electrical system provided within the console of FIG. 1. The system 10′ includes a power supply 22′ disposed in the console 20′ that provides current for the pads 30′ through a set of attenuators 24′ and 26′ for each pad 30′. The first attenuator 24′ regulates current to a set of stimulation contacts 32 and 34 provided on an exposed surface of the pad 30′ and the second attenuator 26′ regulates current to a heating coil 36′ embedded within the pad. A pad select switch 28′ provides an enable signal for each attenuator under operator control and outputs the setting level status to the operator via a display 29′. Note that the system 10′ only sets the heat and stimulation current levels. As no temperature sensor is provided in the conventional pad 30′, no pad temperature regulation or control is possible.

In addition, it should also be noted that the electrical arrangement of FIG. 2 is provided for illustration only. Other electrical arrangements may be known and used in the art.

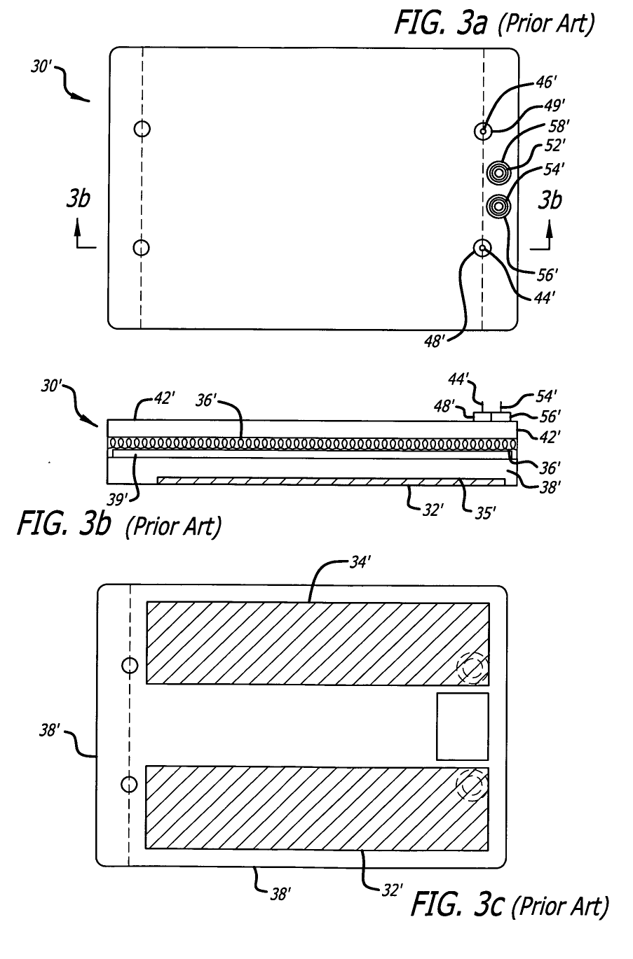

FIGS. 3a-3c illustrate the typical conventional thermostimulation pad 30′ of FIGS. 1 and 2 in more detail. FIG. 3a is a top view of the pad, FIG. 3b is a sectional side view taken along the line b-b of FIG. 3a, and FIG. 3c is a bottom view of the pad. The conventional pads 30′ are fabricated of silicone sheets glued together to encapsulate a foil heater with a separate wire for an electrostimulation pad. The pads are die cut from rolled sheets of silicone and therefore typically have sharp edges that are often uncomfortable to the patient. The upper surface is translucent and the bottom surface is gray silicone.

As best illustrated in the side view of FIG. 3b, the conventional pad 30′ includes the first and second electrically conductive strips 32′ and 34′ for electrostimulation. Shown more clearly in the bottom view of FIG. 3c, the strips 32′ and 34′ are fabricated of carbon loaded silicone (i.e., polymerized siloxane or polysiloxane) or other suitable material. Returning to FIG. 3b, the conductive strips 32′ and 34′ are secured to a pad body 38′ by a layer or pad of glue 35′.

Typically, the pad body 38′ is also fabricated of silicone. A second layer of silicone 39′ is provided on the pad body 38′ for structural support. The upper surface of the second layer 39′ is typically treated with a primer (not shown) and the heating element 36′ is mounted between the primed second layer 39′ and a primed third layer of silicone 42′. The heating element 36′ is typically a coil fabricated with aluminum foil.

Stimulation current is provided via wires 70′ attached to the conductive strips 32′ and 34′ by first and second press stud type connectors 44′ and 46′. Though not shown in FIG. 3b, the connectors 44′ and 46′ extend through the third, second and first structural layers 42′, 39′ and 38′ sequentially to connect with the electrical stimulation strips 32′ and 34′. Silicone covers 48′ and 49′ extend around the upper end of the connectors 44′ and 46′ respectively to protect the patient from electrical burns. In FIG. 3b, the cover 48′ is shown depressed to expose the connector 44′ to illustrate that a wire from the console 20′ may be clipped thereto.

A second set of connectors 52′ and 54′ extend through the third layer 42′ to the heating coil 36′ and provide electrical connectivity thereto. Silicone covers 56′ and 58′ are provided for the second set of connectors 52′ and 54′ respectively.

As noted above, there are several shortcomings associated with the conventional pad design set forth above. That is, conventional pads are hard and die cut with sharp flat edges. The rectangular shape of the pads does not conform to the natural shape of muscle tissue. In addition, conventional pads tend to exhibit a current fall off over the length of the pad. This limits the performance of conventional pads. Further, the connectors are subject to detachment and therefor often fail to comply with government requirements in certain countries i.e., EN standard 60601-2-35 for medical electrical devices. In addition, conventional thermostimulation pads are not waterproof and have recesses into which materials can be deposited which are difficult to clean and could carry risk of infection from patient to patient. As a consequence, sweat from the patient combined with the pad gel can cause the stimulation connector and press studs to short directly to the patient, which can result in the patient being shocked or burned. Moreover, conventional thermostimulation pads are too hard and, being too inflexible, yield to frequent bending and breakage of the coil disposed therein. More significantly, conventional thermostimulation pads are not designed to detect, measure and/or monitor temperature. Hence, a need remains in the art for an improved system or method for thermostimulation therapy that is more safe and effective. As discussed more fully below, the inventive pads address this need in the art.

### Inventive System

Overall System

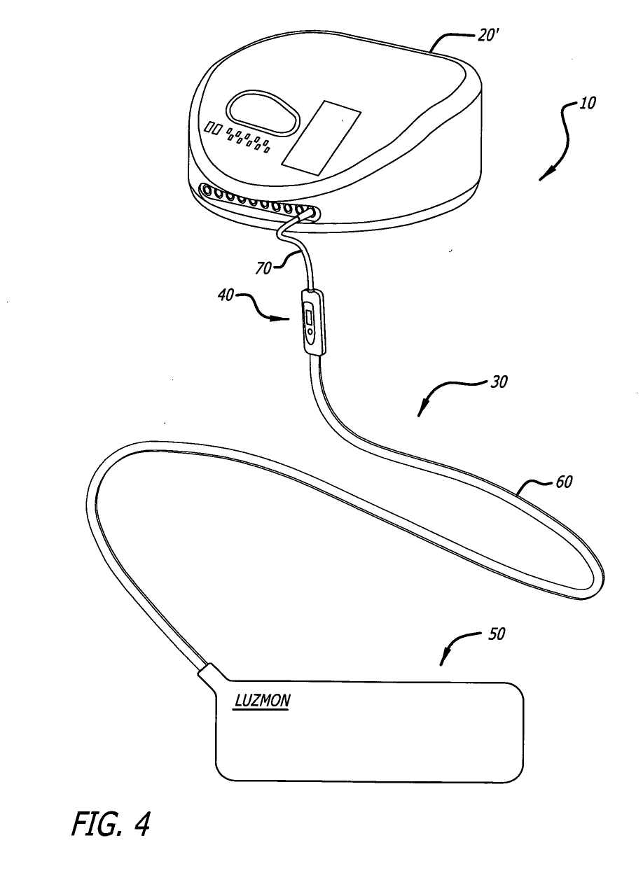

FIG. 4 is a simplified perspective view of a thermostimulation system implemented in accordance with an illustrative embodiment of the present teachings. As shown in FIG. 4, the system 10 includes a conventional thermostimulation console 20′ with, in accordance with the present teachings, a plurality of novel thermostimulation pad assemblies 30 electrically coupled thereto. Each pad assembly 30 includes a novel inline control system 40 and an associated multilayer injection molded dual function (heat and stimulation) pad 50 of unique design and construction with integrated sensor in accordance with the present teachings. Each control system 40 is connected to an associated pad 50 via a cable 60. As discussed more fully below, in the best mode, the cable 60 is flat.

Pads

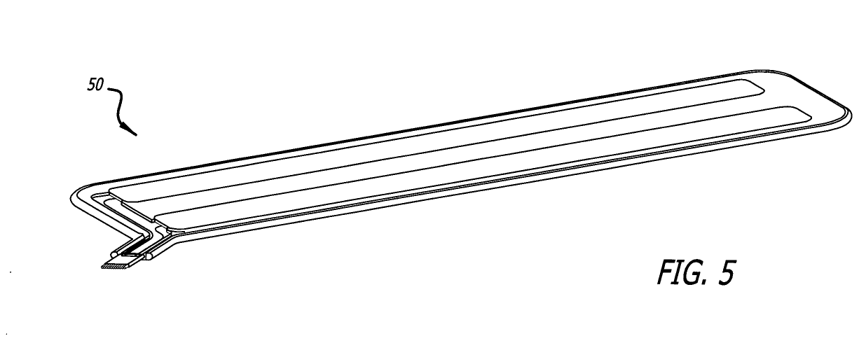

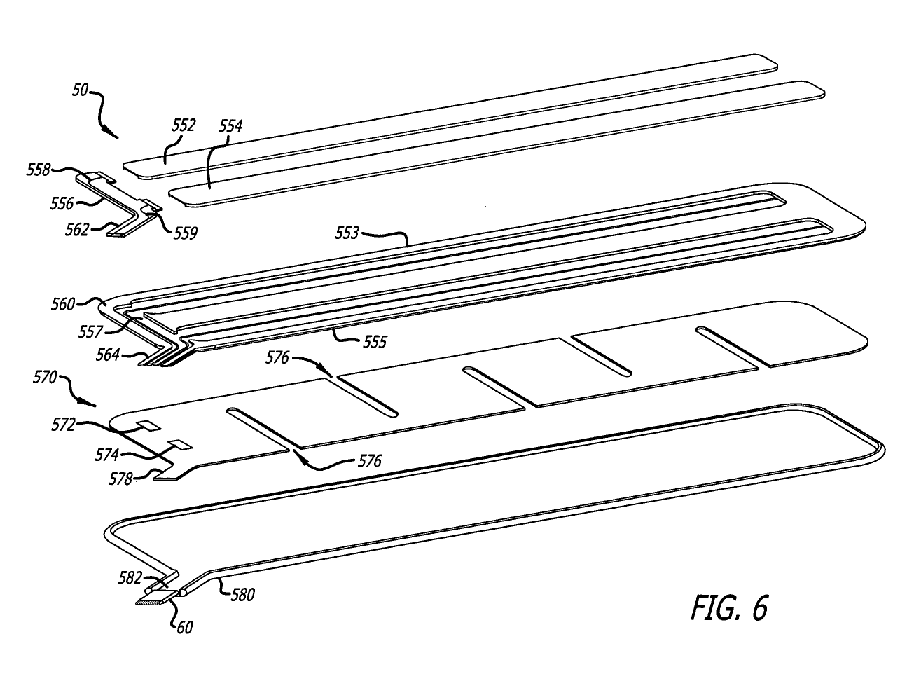

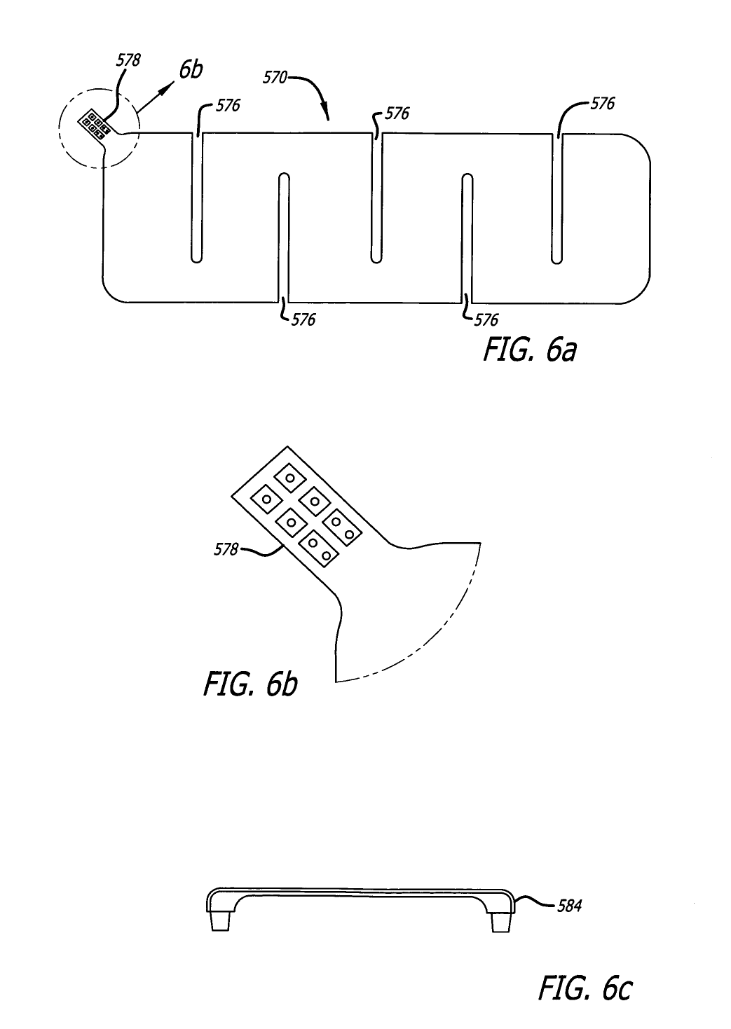

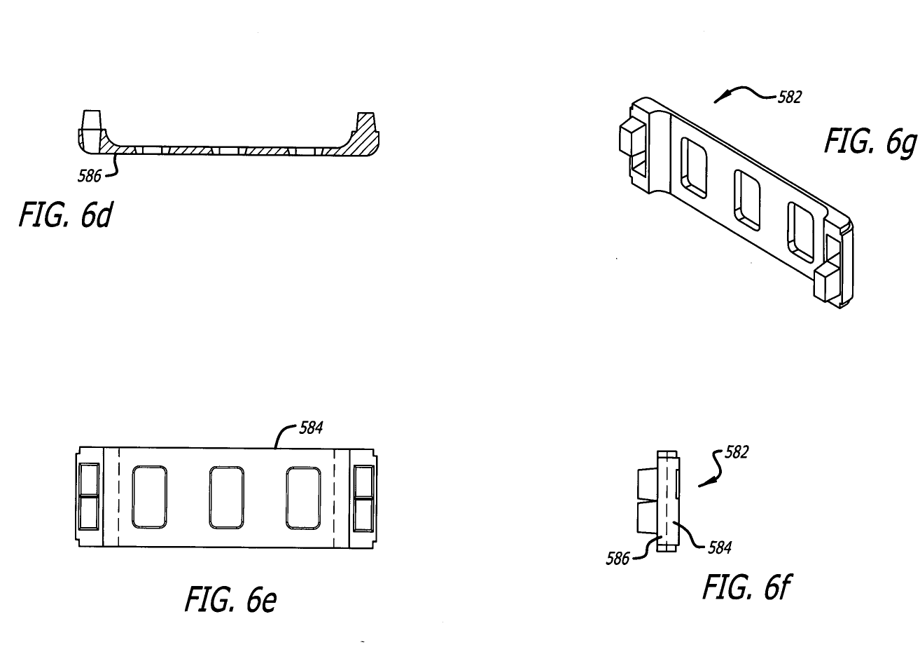

FIG. 5 shows a perspective bottom view of the pad 50 of FIG. 4. FIG. 6 is an exploded upside down view of a portion of the pad 50 of FIG. 4 in disassembled relation. As shown in FIGS. 5 and 6, the pad 50 includes first and second elongate substantially parallel conductive strips 552 and 554. In the illustrative embodiment, each conductive strip has a Shore hardness of 50 A—i.e. medical grade (USB Class 6) ten percent (10%) carbon loaded silicone. For example, Wacker LR 3162 could be used. This product has an electrical resistance of 1 kΩ per cm. In the illustrative embodiment, the strips are 51.5 millimeters (mm) wide, 521 mm in length and 1.85 mm thick. Those of ordinary skill in the art will appreciate that the present teachings are not limited to the dimensions of the illustrative embodiment.

A polymer connector 556 is coupled to one end of the first and second strips 552 and 554 and serves as an end piece therefor and the second end of each strip is free. In the illustrative embodiment, the connector 556 is fabricated of Shore 40 A silicone and serves as an insulator and support for wires 558 and 559 that provide a connection to the strips 552 and 554 respectively. In practice, one of the strips is powered a positive contact and the other provides a negative contact.

The two strips 552 and 554 are molded and then the end piece 556 is molded separately. These pieces are glued together and placed back into a mold and the next layer 560 is over-molded over the assembly to provide a single molded piece consisting of the strips 552, 554, end piece 556, and layer 560. In the preferred embodiment, the over-layer 560 is made of medical grade Shore 40 A polymer or other material suitable for a particular application. Note the grooves 553 and 555 and recess 557 within the over-layer adapted to receive and seat the strips 552 and 554 and the end piece 556 respectively. The wires 558 and 559 are then laid into the slots running through the overmould layer and into the grooves in the stimulation strips 552 and 554. The wires are then glued in place using a carbon loaded RTV (room temperature vulcanized) silicone glue. This allows for the electrical current to be passed from the wires 558 and 559 to the stimulation strips 552 and 554. Once cured, the remaining space in the slot in the 560 overmould layer is filled with non-conductive RTV silicone glue up to the same level of the surface of the overmould layer 560.

As shown in FIG. 6, a heating element 570 is provided over the layer 560. In the best mode, the heating element 570 is implemented as a built in wire matrix and is held in place with a layer of silicone 580. First and second temperature sensors 572 and 574 are mounted in the heating element 570, one is a live sensor measuring temperature and feeding this information back to the control box and the second is a back up should the first sensor fail. In the illustrative embodiment, each temperature sensor is implemented as a conventional 1 kilo-ohm RTD (resistive temperature detector). In the illustrative embodiment, the heating element is a wire matrix bonded in silicone with a thickness of 0.75 mm, over the majority of the surface apart from where the RTDs are mounted, and is rated at 400 watts per square meter using 24 volts alternating current. Note the provision of slots 576 in the heating element 570. These slots serve to improve flexibility in all planes of the element.

In the illustrative embodiment, as illustrated in the top plan view of FIG. 6a and the magnified view of FIG. 6b, the extension 578 of the heating element 570 has a number of solder connections to facilitate electrical connection of the heating element 570 to the cable 60. The extension tab 578 is adapted to be received within a strain relief grommet 582 in the heater over-layer 580 along with the extensions 562 of the end piece 556 and 564 of the layer 560. In the illustrative embodiment, the grommet does not come into contact with the extension 578. The grommet 582 receives the flat cable 60 which is then stripped back and the associated wires are connected to the various solder pads on the extension 578. FIG. 6c shows the upper section 584 of the illustrative implementation of the grommet 582. FIG. 6d shows the lower section 586 of the illustrative implementation of the grommet 582. FIG. 6e is a top view of the grommet, FIG. 6f is a side view of the grommet and FIG. 6g is a perspective view of the upper section of the grommet 582. In the preferred embodiment, the grommet 582 is made of TPU (thermoplastic polyurethane) and is implemented in two halves, an upper section 584 and a lower section 586. The upper and lower sections 584 and 586 are glued together and these sections are glued to the flat cable 60. In the best mode, the upper and lower sections 584 and 586 of the grommet 582 are glued together and to the cable 60 with cyanoacrylate glue.

Those skilled in the art will appreciate that the present invention is not limited to the materials utilized in the fabrication of the illustrative embodiment. Other materials may be used without departing from the scope of the present teachings.

In the illustrative embodiment, the heater over-layer 580 is Shore 40 A medical grade silicone in construction. Nonetheless, as noted above, it should be noted that the present invention is not limited to any particular material or hardness.

Each pad is assembled from the stimulation side. In the best mode, the structure of the pad 50 is based on a multi-step injection molding process, with over-molding of the various layers to build up the base of the pad to the complete pad thickness and embed and encapsulate the various components within it, such as the electrostimulation wires and heating element. The final step is to insert and bond the top lid of the pad into the assembled structure. The steps of the injection molding process include moulding of the stimulation strips, over moulding of the stimulation strips to encapsulate the stimulation wires to create the patient facing surface of the pad and the moulding of the lid of the pad 580 which encapsulates the heating element and creates the upper facing surface of the pad and seals in the flat cable and grommet.

Hence, in accordance with the present teachings, the strips 552 and 554 and the layers 560, 570 and 580 and the grommet 582 are molded into a single unitary multilayer injection molded dual function (heat and electrostimulation) construction.

Flat Cables

Returning to FIG. 4, a novel flat cable 60 connects each pad to its associated inline control and a conventional cable 70 is used to connect each inline control system to the console 20. The flat cables 60 enhance patient comfort. In the illustrative embodiment, the flat cable 60 linking the control system 40 to the pad 50 is approximately 2.5 meters long and 17.38 mm wide. The cable 60 has inner core of 14 insulated wires, with an outer protective sheath in a white polyvinyl chloride (PVC). Flat cables are commercially available from manufacturers such as Spectra Strip of Hampshire, Great Britain.

Inline Control System

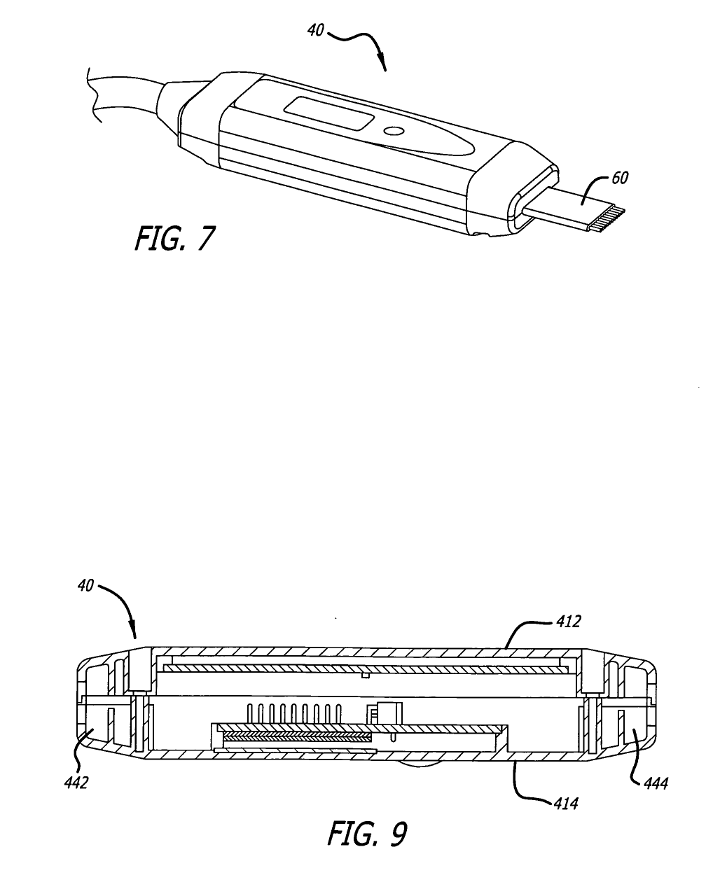

FIG. 7 is a perspective side view of the inline control system 40 of FIG. 4 fully assembled.

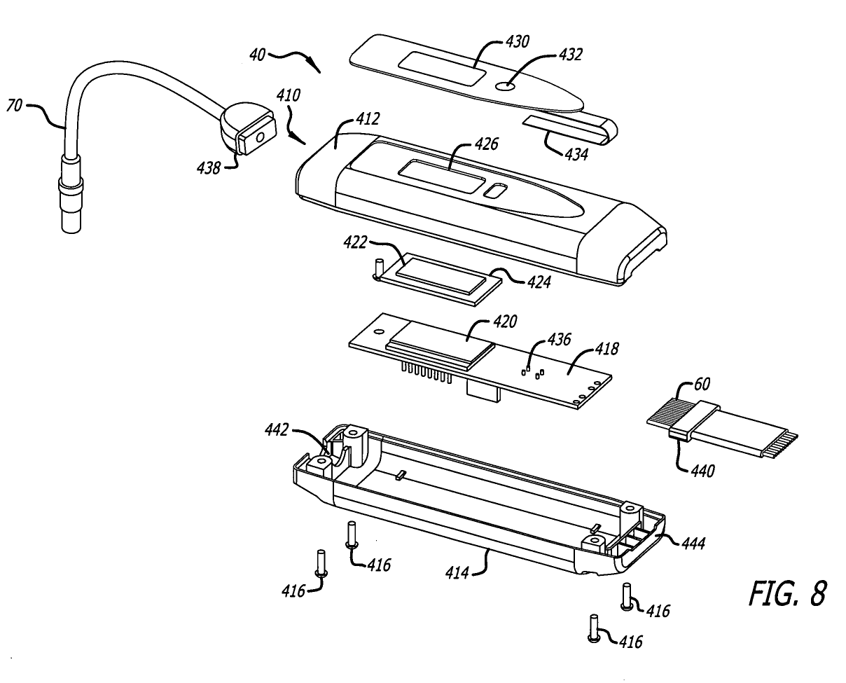

FIG. 8 is a perspective side view of the inline control system 40 of FIG. 7 disassembled. As shown in FIG. 8, the control system 40 includes a two part injected molded ABS plastic housing 410 with an upper casing 412 and a lower casing 414. The housing 410 is adapted to retain a multilayer printed circuit board 418 on which an integrated circuit 420 is disposed. A microprocessor (not shown) is provided by the integrated circuit 420. Numerous additional electrical components are mounted onto the printed circuit board 418 along with a liquid crystal display (LCD) 422.

As shown in FIG. 8, a small Perspex window 430 protects the LCD 422. In the best mode, the LCD display 422 shows both the target and actual temperatures for the associated pad. The window 430 seats within an aperture 426 in the upper casing 412 of the housing 410. A plate 430 contoured to fit within a depression on the upper surface of the upper casing 412 is fitted with a manual override switch 432. The switch 432 connects to the control circuitry on the printed circuit board 418 via a flexible wire 434 and pins 436.

In the illustrative embodiment, a switch is used to enable the user to confirm when a user wants to heat a pad 50 above 38 degrees Celsius. The round cable 70 coming from the console 20 enters the top of the control system 40 and is held in place by a second grommet 438. The flat cable 60 enters the system 40 from the bottom and is held in place by a third grommet 440 that is also used as a strain relief device at the cable termination with the pad. In the illustrative embodiment, this grommet is a standard, over-the-counter cable retention fixator. As discussed more fully below, the third grommet 440 is a two section grommet which captures the flat cable as it enters the system 40. The system 40 is then held together by four screws 416. As illustrated in the sectional side view of FIG. 9, when secured together, the upper and lower casings 412 and 414 provide first and second chambers for seating the second grommet 438 and the third grommet 440.

Electronics

As mentioned above, each pad has a heating element, two RTD sensors (one for active temperature control and another for backup) and two stimulation pads that make electrical contact with the user.

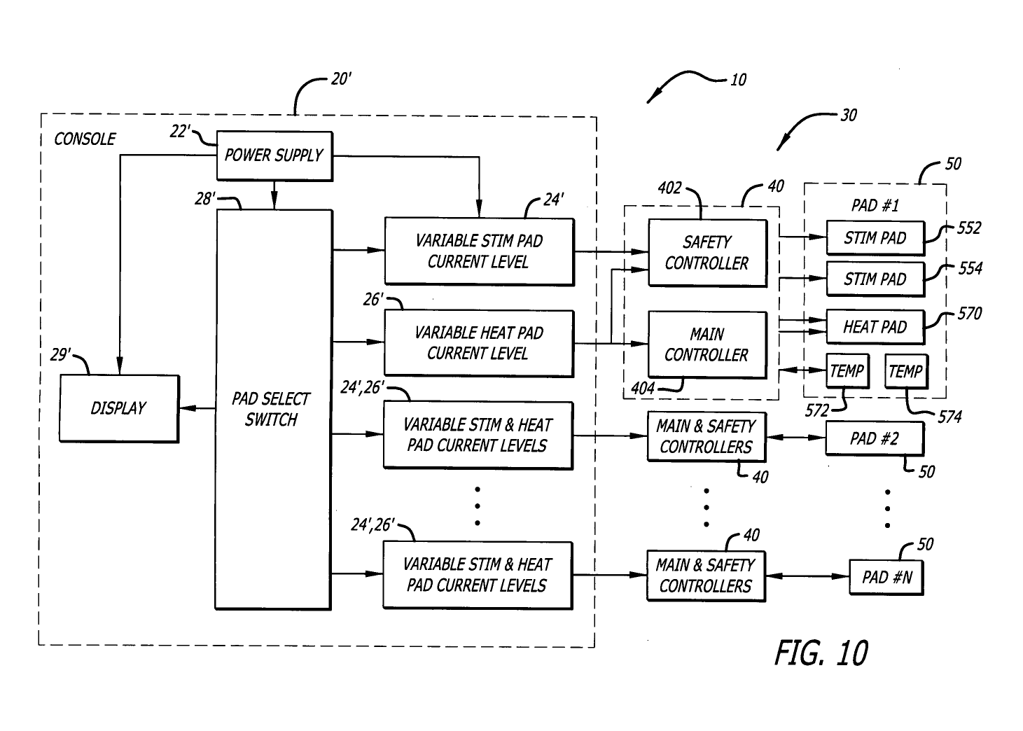

FIG. 10 is an electrical block diagram of the inventive system 10 including the control system elements 40. The circuitry of the control system 40 is powered by the heating current from the console 20. The control system 40 provides intelligent operation for the pad 50, monitoring the current going to both electrostimulation pads 552 and 554 and the heating element 570. These currents can be set at different levels by the control system 40 depending on the program selected or manually adjusted after a program is selected. The conventional console 20 does not allow for the temperature to be measured or monitored but instead typically has a heating current level setting described as a “heating percentage”. Since a regulation or control functionality is not conventionally available, the current sent to the pads could allow them to heat to more than 42 degrees Celsius, a level which is outside of safe levels and the requirements set by the EN60601-2-35 standard.

As illustrated in FIG. 10, in the illustrative embodiment, each control system 40 is implemented with first and second microcontrollers (implemented in the best mode with microprocessors) 404 and 402, that control and interrupt the current to the stimulation electrodes 552 and 554 and the heating element 570 of FIG. 6 respectively. The first controller 404 serves as a main controller and the second controller 402 serves as a safety controller. As discussed more fully below, each microcontroller runs unique software (i.e. firmware) stored on a tangible medium, such as an electrically erasable programmable read only memory (EPROM), in the integrated circuit 420 of FIG. 8.

Software

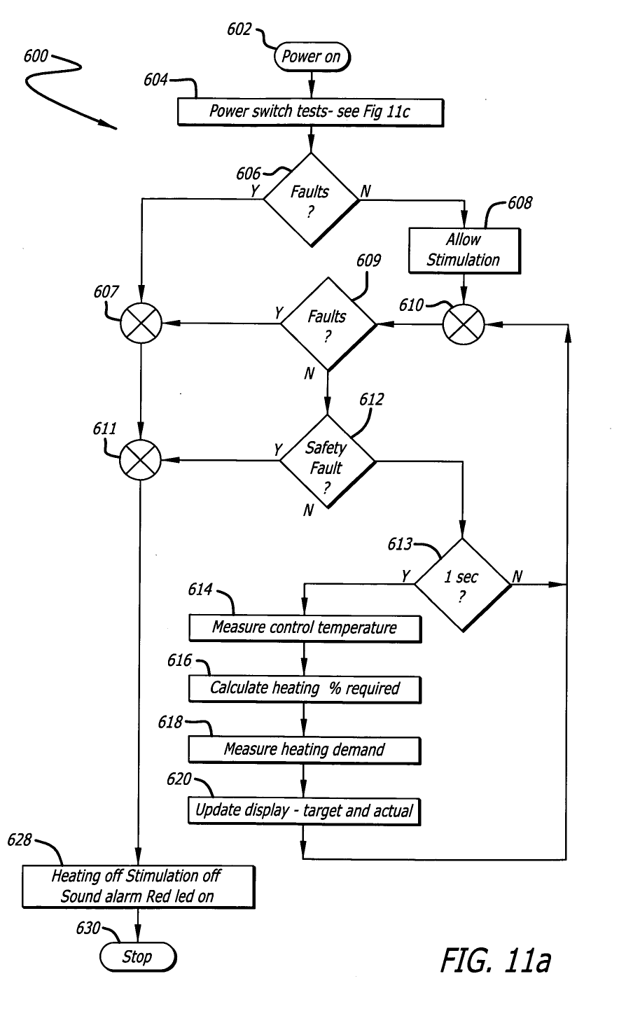

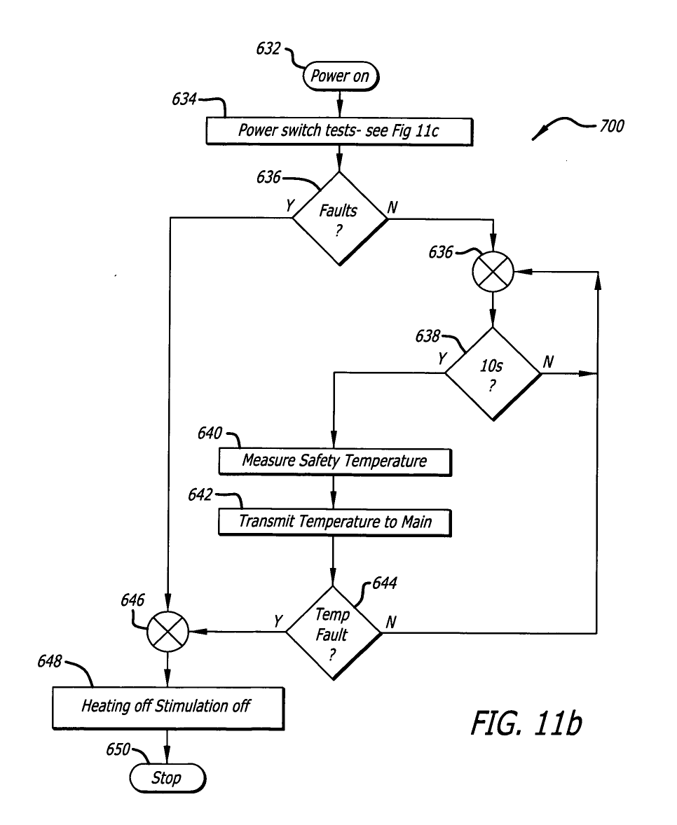

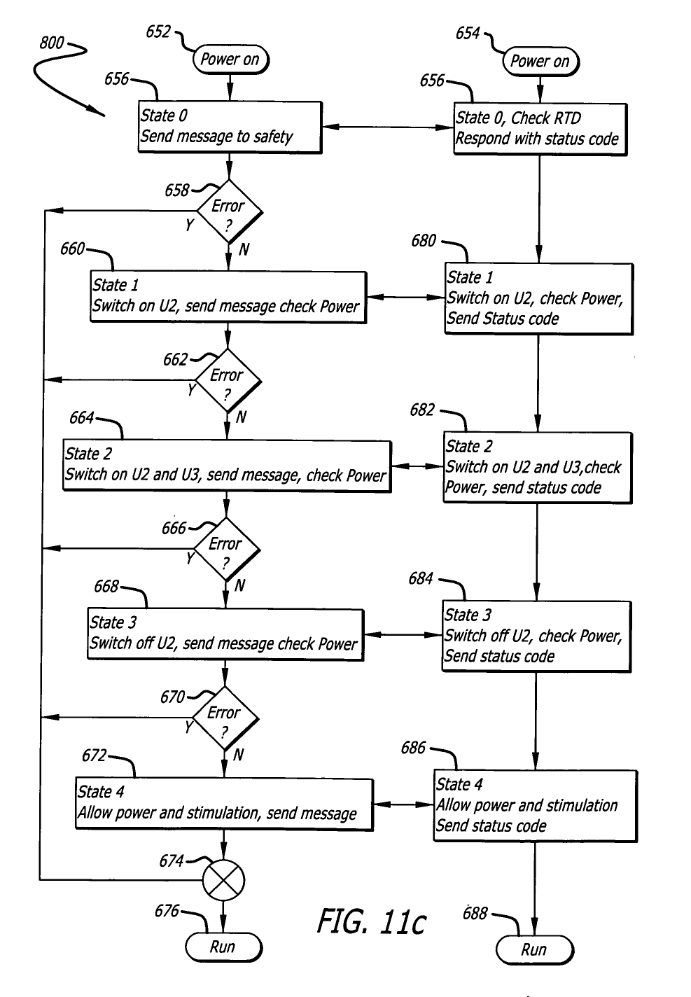

FIGS. 11a-c are flow diagrams of the firmware executed by the microprocessors in accordance with an illustrative embodiment of the present teachings. FIG. 11a is a flow diagram of the firmware executed by the main microcontroller 402 of FIG. 10. FIG. 11b is a flow diagram of the firmware executed by the safety microcontroller 402 of FIG. 10. FIG. 11c is a flow diagram of the firmware executed by the main and safety microcontrollers of FIG. 10 for a self-test mode of operation. Both microcontrollers monitor the heating power control devices to determine whether they perform the correct on-off switching action or have failed as a short circuit or an open circuit. During the power up stage, the MMC and SMC communicate using an asynchronous communications link. In the illustrative embodiment, the microprocessors communicate with each other every second to pass status information using an I2C serial interface.

The MMC 404 sends messages to the SMC to tell it which test is being performed and then the SMC 402 sends the results of the tests at each stage. Only if all the stages pass with no failures is power applied to the heating circuit 570 in the pad.

During power up (602), or at a power setting greater than five percent (5%) of maximum, the main microcontroller (MMC) 404 performs a self-test (604) to detect any possible failures and then communicates with the safety microcontroller (SMC) 402. As illustrated in FIG. 11c, the self-tests are synchronised such that all hardware functionality is tested before enabling heating power to the patient.

The pad assembly, including the electronics, is calibrated. Calibration information is stored in an EPROM (not shown) within the MMC 404. In order that the SMC 402 can accurately determine whether the associated regulated pad is overheating, a calibrated maximum temperature value is passed from the MMC to the SMC during the power up procedure.

After checking for faults (606) the MMC 404 enables stimulation (608) and monitors the percentage power setting of the console 20 (see steps 614-616). This is used to set a target temperature for the pad. This target temperature is displayed on the LCD 422. Should the target temperature be greater than 38° C. the software 600 requires the operator to press the front panel switch on the console 20 to confirm the intention to set a higher temperature. Table I below lists illustrative target temperatures corresponding to various power levels.

TABLE I

| Power setting % | Target Temperature |
|---|---|
| 5-20 | 36 |
| 20-30 | 37 |
| 30-40 | 38 |
| 40-50 | 39 |
| 50-60 | 40 |
| 60-100 | 41 |

In the illustrative embodiment, a reduction in target temperature would not have to be confirmed.

During the pre-heating stage of a procedure the CTEMS unit demand 100% heat for three minutes. This is to heat up the pads prior to placement on a patient. This is interpreted as a demand for 41° C. and if this temperature is not confirmed by the operator the unit will heat up to the safety temperature of 38° C.

The MMC controls the temperature using a PID control loop. The actual temperature is measured using the temperature sensor 572 embedded in the pad. The SMC monitors the pad temperature using the other temperature sensor 574.

There is a two colour LED in the front facing section of the connection box. This will flash red and green and is used to provide status information.

TABLE II

| LED | Information |
|---|---|
| Green flashing | Heating up to target |
| Green continuous | Target reached |
| Red flashing | Over temperature, when actual is above |
| | target but not above 42° C. |
| Red continuous | Fault, heating and stimulation disabled. This |
| | could be temperature above 42° C. or a |
| | hardware fault. |

As shown in FIG. 11b, after performing self-tests (634) the SMC 402 measures the safety temperature via the second sensor 574 and disables the associated pad 50 if the specified maximum temperature is reached or exceeded.

Operation

The following describes the method of operation of the inventive system 10 in accordance with an illustrative implementation thereof:

1. First, the operator plugs the pad cord 70 into the front of the console 20.

2. Next, the operator selects the desired program and starts the preheating phase. The display will flash at 41° C. and then heat up to 38° C. unless the override button 432 (FIG. 8) is pressed at which stage it would heat to 41° C. The preheating phase lasts for 3 minutes (and can be repeated).

3. Once the preheating is complete, the user presses “pause” on the system itself and the LCD display on the connection box will go blank.

4. The patient is laid on the bed and the pads are strapped to the patient in the desired configuration.

5. The operator then presses pause again and the program starts. The LCD 422 will then show the actual and target temperature again and the user will have to press the membrane button on each pad if the system program has a current % of 40% or above to allow the pad to heat to above 38° C.

6. The pad control system 40 will then monitor the temperature and ensure that it does not go above the desired level.

7. If the LED goes continually red for a period of more than a couple of minutes pad control system 40 will interrupt all the currents (both electrical stimulation and heat) to the pad and the operator will put the system on pause and replace the pad.

Thus, the present invention has been described herein with reference to a particular embodiment for a particular application. Those having ordinary skill in the art and access to the present teachings will recognize additional modifications, applications and embodiments within the scope thereof.

It is therefore intended by the appended claims to cover any and all such applications, modifications and embodiments within the scope of the present invention.

Accordingly,

What is claimed is:

1. A thermostimulation system for use with a console for providing electrical currents for thermal and electrical stimulation in response to a first input from an operator via at least one electrical connector, said thermostimulation system comprising:
an inline control system coupled to said electrical connector for regulating said currents in response to a second input from an operator and a temperature feedback signal via a second electrical connector and
a thermostimulation pad coupled to said inline control system via said second electrical connector, said pad having a temperature sensor adapted to provide said feedback signal to said inline control system.

2. The invention of claim 1 wherein said inline control system includes a first control means having heat control means for regulating heating current to said pad.

3. The invention of claim 2 wherein said first control system includes stimulation control means for regulating stimulation current to said pad.

4. The invention of claim 2 wherein said inline control system includes a first microprocessor.

5. The invention of claim 4 wherein said inline control system includes first firmware stored in a physical medium in said control system and adapted for execution by said microprocessor.

6. The invention of claim 2 wherein said inline control system includes a second control means for interrupting current flow to said pad on receipt of a temperature signal from said pad that exceeds a predetermined first threshold.

7. The invention of claim 6 wherein said second control means is adapted to regulate heating current to said pad.

8. The invention of claim 7 wherein said second control means is adapted to regulate stimulation current to said pad.

9. The invention of claim 6 wherein said inline control system includes first and second microprocessors.

10. The invention of claim 9 wherein said inline control system includes first and second firmware stored in a physical medium in said control means and adapted for execution by said first and second microprocessors.

11. The invention of claim 1 wherein said inline control system includes a display.

12. The invention of claim 1 wherein said inline control system includes a patient cutoff switch.

13. The invention of claim 1 wherein said pad has a heating element consisting of a wire matrix.

14. The invention of claim 13 wherein said heating element is slotted.

15. The invention of claim 1 wherein said pad has at least one electrical stimulation contact having a conductor along the length thereof.

16. The invention of claim 15 wherein said pad has two electrical stimulation contacts each having a conductor along the length thereof.

17. The invention of claim 1 wherein said pad has an integrated multilayer construction.

18. The invention of claim 1 wherein said pad further includes a strain relief grommet for the connection thereof to said second connector.

19. The invention of claim 1 wherein said second connector is substantially planar.

20. The invention of claim 1 wherein said system includes a plurality of pads with associated inline control systems electrically coupled to said console.

21. A thermostimulation system comprising:
a console for providing electrical currents for thermal and electrical stimulation in response to a first input from an operator via at least one electrical connector;
a plurality of inline control systems coupled to said electrical connector for regulating said currents in response to a second input from an operator and a temperature feedback signal via a second electrical connector, each of said inline control systems having heat control means and stimulation control means; and
a thermostimulation pad coupled to each of said inline control systems via a respective one of said second electrical connectors, each of said pads having an integrated multilayer construction and a temperature sensor adapted to provide said feedback signal to a respective one of said inline control systems.

22. The invention of claim 21 wherein each of said inline control systems includes a first microprocessor.

23. The invention of claim 22 wherein each of said inline control system includes first firmware stored in a physical medium in each of said control means and adapted for execution by said microprocessor.

24. The invention of claim 21 wherein each of said inline control systems includes means for interrupting current flow to said pad on receipt of a temperature signal from said pad that exceeds a predetermined first threshold.

25. The invention of claim 24 wherein said second means for interrupting current flow to said pad is adapted to regulate heating current to said pad.

26. The invention of claim 25 wherein said means for interrupting current flow to said pad is adapted to regulate stimulation current to said pad.

27. The invention of claim 21 wherein said inline control system includes first and second microprocessors.

28. The invention of claim 27 wherein said inline control system includes first and second firmware stored in a physical medium in said control means and adapted for execution by said first and second microprocessors.

29. The invention of claim 21 wherein said inline control system includes a display.

30. The invention of claim 21 wherein said inline control system includes a patient cutoff switch.

31. The invention of claim 21 wherein said pad has a heating element consisting of a wire matrix.

32. The invention of claim 31 wherein said heating element is slotted.

33. The invention of claim 21 wherein said pad has at least one electrical stimulation contact having a conductor along the length thereof.

34. The invention of claim 33 wherein said pad has two electrical stimulation contacts each having a conductor along the length thereof.

35. The invention of claim 21 wherein said pad further includes a strain relief grommet for the connection thereof to said second connector.

36. The invention of claim 21 wherein said second connector is substantially planar.

37. A thermostimulation method including the steps of:
applying a thermostimulation pad with connector integrated multilayer construction to a patient having a temperature sensor adapted to feedback a temperature signal;
coupling said pad to a console via an inline control system;
setting said console to generate predetermined electrical currents to the inline control system for thermal and electrical stimulation via a first connector; and
regulating the temperature of said pad via the inline control system in response to said predetermined electrical current for thermal stimulation and said feedback temperature signal.
